(19) United States
(12) Patent Application Publication
(10) Pub. No.: US 2006/0247641 A1
(43) Pub. Date: Nov. 2, 2006

(54) Method and apparatus for the repair of a rotator cuff (RTC) tendon or ligament

(76) Inventors: Paul Re, Boston, MA (US); Louis P. Re, Boston, MA (US); Peter F. Marshall, Bolton, MA (US); Karl Reese, Boston, MA (US); Dennis Hubbard, Lancaster, MA (US)

Correspondence Address: Mark J. Pandiscio;Pandiscio & Pandiscio, 470 Totten Pond Road, Waltham, MA, 02451-1914, US

(21) Appl. No.: 11/280,600

(22) Filed: Nov. 15, 2005

Related U.S. Application Data
(60) Provisional application No. 60/628,082, filed on Nov. 15, 2004.

(51) Int. Cl. A61B 17/58

(52) U.S. Cl. 606/72

(57) ABSTRACT

Apparatus for securing soft tissue to bone, comprising: a suture anchor comprising: a body; an elongated tip section connected to the body and extending distally of the body, the elongated tip section terminating in a distal point; a bone-engaging geometry formed on the body, the bone-engaging geometry being proximal to the elongated tip section; a driver-engaging element formed on the proximal end of the body; and a suture-connecting feature associated with the body for attaching a suture to the body; wherein the elongated tip section has a length which exceeds the thickness of the soft tissue which is to be secured to the bone, such that when the suture anchor is passed through the soft tissue, the distal point emerges from the soft tissue before the bone-engaging geometry penetrates the soft tissue.

### REFERENCE TO PENDING PRIOR PATENT APPLICATION

This patent application claims benefit of pending prior U.S. Patent Application Ser. No. 60/628,082, filed Nov. 15, 2004 by Paul Re et al. for METHOD AND APPARATUS FOR THE REPAIR OF A ROTATOR CUFF TENDON OR LIGAMENT (Attorney's Docket No. RE-3 PROV), which patent application is hereby incorporated herein by reference.

### FIELD OF THE INVENTION

This invention relates to surgical methods and apparatus in general, and more particularly to methods and apparatus for repairing a rotator cuff (RTC) tendon or ligament.

### BACKGROUND OF THE INVENTION

A tendon is the fibrous tissue which connects a muscle to a bone, thereby allowing the muscle to exert its force on the bone, e.g., at a joint.

Tendons are frequently damaged (e.g., detached, torn, ruptured, etc.) as the result of injury, wear and tear, and/or accident. A damaged tendon can impede proper articulation of a joint, and/or cause weakness, dyskinesis, arthritis and/or pain.

Among the tendons which are most frequently damaged are those attached to the muscles surrounding the shoulder joint (i.e., the humeral head). These tendons and their associated muscles are commonly referred to as the rotator cuff (RTC). The rotator cuff (RTC) tendon which is most commonly damaged is the supraspinatus tendon.

More particularly, and looking now at FIG. 1 there is shown an intact rotator cuff (RTC) tendon insertion onto the insertion site (commonly called the “footprint”) of the humeral head. In other words, there is shown a rotator cuff (RTC) tendon 5 which is fully connected to the humeral head 10, as would be expected with a normal, undamaged rotator cuff (RTC) tendon.

In FIG. 2, the rotator cuff (RTC) is shown completely torn away from the humeral head footprint. In other words, rotator cuff (RTC) 5 is shown fully detached from humeral head 10, which exemplifies one common form of rotator cuff (RTC) damage.

In FIG. 3, the rotator cuff (RTC) is shown partially torn away from the humeral head footprint (i.e., a “partial tear”). In other words, there is shown a rotator cuff (RTC) 5 which is partially detached from humeral head 10, which is another common form of rotator cuff (RTC) damage.

Numerous procedures have been developed to repair a damaged rotator cuff (RTC) tendon.

Initially these procedures involved making a large incision into the shoulder, splitting the deltoid muscle, detaching it, and then repairing the torn rotator cuff (RTC) tendon by suturing the tendon back down to the footprint site using drill holes 15, bone tunnels 17 and sutures 20 (FIG. 4). While generally effective, the downsides of this approach are the large size of the incision, the pain caused by the procedure, the technical effort of suture fixation, and the possibility of significant deltoid dysfunction due to splitting, detachment etc.

These disadvantages lead to the development of suture anchors which could be quickly and easily deployed into the bone, thereby providing a simple way to secure sutures (and hence the tendon) to the bone without having to drill holes 15, form bone tunnels 17, pass the sutures 20, etc. The development of suture anchors allowed the procedure to be done with a significantly smaller incision, less pain for the patient, less trauma to the tissue, reduced risk of significant deltoid damage, and greater speed and convenience for the surgeon.

An example of one such suture anchor is shown in FIG. 5. More particularly, and looking now at FIG. 5, there is shown a suture anchor 25 which generally comprise a body 30 having a tip section 31 terminating in a distal point 33 to facilitate introducing the suture anchor into a bone, screw threads 35 for advancing the suture anchor into the interior of the bone, a hex-shaped rear end 40 for coupling the suture anchor to a rotary driver (not shown in FIG. 5), and an eyelet 45 for attaching a suture 50 to the suture anchor.

A next step in the evolution of rotator cuff (RTC) tendon repair was the transition to performing the rotator cuff (RTC) tendon repair as an arthroscopic (or “minimally invasive”) procedure. Such an arthroscopic rotator cuff (RTC) tendon repair generally utilizes three or more small (e.g., 5 mm) incisions, typically called “portals”. A small (e.g., 3.5 mm) camera (commonly called an “arthroscope”) is typically deployed through one portal to provide visualization of the interior of the shoulder. The remaining portals are then used to introduce microinstruments into the interior of the shoulder to perform the rotator cuff (RTC) tendon repair. Although technically more demanding, this arthroscopic procedure is less painful for the patient, less damaging to the deltoid muscle, and allows for a faster recovery.

An example of such an arthoscopic rotator cuff (RTC) tendon repair is shown in FIGS. 6-11. This example shows an arthroscopic procedure to repair a rotator cuff (RTC) tendon 5 which has been torn completely away from the humeral head 10. More particularly, this arthroscopic procedure generally involves grasping the damaged rotator cuff (RTC) tendon 5 with a grasper instrument 55 (FIG. 7), pulling the tendon laterally back into position over the footprint on the humeral head, and then holding the rotator cuff (RTC) tendon in that position while the tendon is re-attached to humeral head 10 at the footprint. This is done by introducing a suture anchor 25 (and its associated inserter 52) into the operative field through another portal, and then advancing the suture anchor 25 through the rotator cuff (RTC) tendon 5 and into the bone 10 using the inserter 52 (FIGS. 8-10). It should be noted that as this is done, due to the way the rotator cuff (RTC) tendon directly overlies the underlying bone, the surgeon is unable to directly visualize the tip of the suture anchor as the tip of the suture anchor emerges from the underside of the rotator cuff (RTC) tendon and enters the bone. Once suture anchor 25 has been advanced through rotator cuff (RTC) tendon 5 and into bone 10, sutures 50 are tied so as to secure rotator cuff (RTC) tendon 5 to bone 10 (FIG. 11).

In general, it is far more preferable to perform rotator cuff (RTC) tendon repairs arthroscopically rather than with an open procedure, inasmuch as the arthroscopic procedure is significantly less painful for the patient, causes less damage to other shoulder structures, and allows for a faster recovery.

However, current suture anchors and methods of their use generally require (i) the use of an additional instrument (i.e., the grasping instrument 55) to re-approximate the rotator cuff (RTC) tendon against the humeral head, and (ii) a “blind” exit of the suture anchor out of the tendon and into the bone.

Unfortunately, the need for an additional instrument (i.e., the grasping instrument 55) may necessitate the use of “another hand” in the operating room, which may not always be readily available.

Furthermore, the “blind” exit of the suture anchor out of the tendon and into the bone can create additional difficulties. More specifically, in the case of a partial thickness rotator cuff (RTC) tendon tear, and in particular an undersurface tear, where it may be more critical for the surgeon to visualize exactly where the suture anchor emerges from the underside of the rotator cuff (RTC) tendon and enters the bone, current anchor designs require that the threads 35 of the suture anchor be directly engaged in the rotator cuff (RTC) tendon (i.e., the surgeon is “committed” in the sense that the screw threads 35 form a relatively large opening in the tendon) (FIG. 12) before the surgeon can see the tip of the suture anchor emerge from the underside of the rotator cuff (RTC) tendon or is able to seat the suture anchor against the underlying bone.

Thus, there is a need for a new and improved method and apparatus for securing a rotator cuff (RTC) tendon against the humeral head.

### SUMMARY OF THE INVENTION

The present invention provides a new and improved method and apparatus for securing a tendon or ligament to a host bone.

More particularly, the present invention provides a novel suture anchor and a novel method for re-attaching a tendon or ligament to bone using that novel suture anchor.

Even more particularly, the present invention comprises the provision and use of a novel suture anchor wherein the tip section of the suture anchor is significantly longer than normal, with a significantly increased distance between the distal point of the suture anchor and the start of the suture anchor's screw threads. In one preferred form of the invention, the tip section of the suture anchor is formed long enough such that the distal point of the suture anchor can be passed through a partially torn rotator cuff (RTC) tendon, or the full thickness of the rotator cuff (RTC) tendon, so that the distal point of the suture anchor can be seen protruding through the undersurface of the rotator cuff (RTC) tendon before the screw threads of the suture anchor have engaged the tendon or the bursal surface of the rotator cuff (RTC) tendon. For reference, it should be noted that the normal thickness of the terminal 2 cm of an intact rotator cuff (RTC) tendon generally ranges from between about 9 mm to about 12 mm in length. Thus, the new suture anchor of the present invention will preferably, but not necessarily, have a tip section (i.e., the distance between distal tip 33 and the start of screw threads 35) which is approximately 10 mm to 20 mm in length.

The present invention also comprises a method of using the elongated tip section of the new suture anchor to spear the rotator cuff (RTC) tendon in such a way that the tendon can be dragged or repositioned or moved or otherwise re-approximated laterally back to the repair site (i.e., to the footprint on the humeral head) and then reseated to the bone without the need for a grasper instrument. In addition, the distal point of the suture anchor can be used as a “starting awl” or punch to aid in the placement of the suture anchor through the rotator cuff (RTC) tendon and into the bone (i.e., the humeral head).

In one form of the present invention, there is provided apparatus for securing soft tissue to bone, comprising:

a suture anchor comprising:
a body;
an elongated tip section connected to the body and extending distally of the body, the elongated tip section terminating in a distal point;
a bone-engaging geometry formed on the body, the bone-engaging geometry being proximal to the elongated tip section;
a driver-engaging element formed on the proximal end of the body; and
a suture-connecting feature associated with the body for attaching a suture to the body;
wherein the elongated tip section has a length which exceeds the thickness of the soft tissue which is to be secured to the bone, such that when the suture anchor is passed through the soft tissue, the distal point emerges from the soft tissue before the bone-engaging geometry penetrates the soft tissue.

In another form of the present invention, there is provided apparatus for securing soft tissue to bone, comprising:

a bone-preparation device having structure for forming a seat in a bone, the bone-preparation device having an axial bore; and

a wire trocar selectively received within the axial bore, the wire trocar comprising a distal point;

wherein, when the wire trocar is received within the bone-preparation device, the distance between the distal point and the distal end of the structure for forming a seat in a bone exceeds the thickness of the soft tissue which is to be secured to the bone.

In another form of the present invention, there is provided a method for securing soft tissue to bone, comprising:

providing apparatus for securing soft tissue to bone, comprising:
a suture anchor comprising:
   a body;
   an elongated tip section connected to the body and extending distally of the body, the elongated tip section terminating in a distal point;
   a bone-engaging geometry formed on the body, the bone-engaging geometry being proximal to the elongated tip section;
   a driver-engaging element formed on the proximal end of the body; and
   a suture-connecting feature associated with the body for attaching a suture to the body;
   wherein the elongated tip section has a length which exceeds the thickness of the soft tissue which is to be secured to the bone, such that when the suture anchor is passed through the soft tissue, the distal point emerges from the soft tissue before the bone-engaging geometry penetrates the soft tissue;

advancing the suture anchor through the soft tissue so that the distal point emerges from the underside of the soft tissue before the bone-engaging geometry engages the soft tissue; and

advancing the suture anchor into the bone.

In another form of the present invention, there is provided a method for securing soft tissue to bone, comprising:

providing:
apparatus for securing soft tissue to bone, comprising:
   a bone-preparation device having structure for forming a seat in a bone, the bone-preparation device having an axial bore;
   a wire trocar selectively received within the axial bore, the wire trocar comprising a distal point;
   wherein, when the wire trocar is received within the bone-preparation device, the distance between the distal point and the distal end of the structure for forming a seat in a bone exceeds the thickness of the soft tissue which is to be secured to the bone; and
   an implant body comprising:
      a body having an axial bore;
      a bone-engaging geometry formed on the body;
      a driver-engaging element formed on the proximal end of the body; and
      a suture-connecting feature associated with the body for attaching a suture to the body;

loading the bone-preparation device onto the wire trocar so that the distance between the distal point and the distal end of the structure for forming a seat in a bone exceeds the thickness of the soft tissue which is to be secured to the bone;

advancing the wire trocar through the soft tissue so that the distal point emerges from the underside of the soft tissue before the structure for forming a seat in the bone engages the soft tissue;

advancing the wire trocar into the bone, and advancing the bone-preparation device into the bone;

withdrawing the bone-preparation device from the bone;

loading the implant body onto the wire trocar and advancing it into the bone; and

withdrawing the wire trocar from the bone.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view of a proximal humerus, showing an intact insertion of the rotator cuff (RTC) tendon onto its insertion site (or “footprint”);

FIG. 2 is a schematic view of a proximal humerus, showing a completely torn or avulsed rotator cuff (RTC) tendon displaced from its insertion site (or footprint);

FIG. 3 is a schematic view of a proximal humerus, showing a rotator cuff (RTC) tendon partially torn from its insertion site (or footprint);

FIG. 4 is a schematic view of a proximal humerus, showing the rotator cuff (RTC) tendon re-attached to its insertion site (or footprint) using drill holes, bone tunnels and suture;

FIG. 5 is a schematic view of a typical prior art rotator cuff (RTC) suture anchor;

FIGS. 6-11 are schematic views showing a torn rotator cuff (RTC) tendon being re-attached to a humerus using a prior art suture anchor technique;

FIG. 12 is a schematic view showing a partially torn rotator cuff (RTC) tendon being re-attached to the humerus using a prior art suture anchor technique;

FIG. 13 is a schematic view showing a novel suture anchor formed in accordance with the present invention;

FIGS. 14-19 are schematic views showing a novel method for re-attaching a completely detached tendon to a bone using the novel suture anchor of FIG. 13;

FIGS. 20-24 are schematic views showing a novel method for re-attaching a partially torn tendon to a bone using the novel suture anchor of FIG. 13;

FIGS. 25 and 26 are schematic views showing a novel method for re-attaching a tendon to bone using the novel suture anchor of FIG. 13 and also a button or similar locking mechanism that is slid down the suture and then tied in place or locked down; and

FIGS. 27 and 28 show additional button or similar locking mechanisms that may be used to hold the soft tissue to the bone;

FIG. 29 is a schematic view showing the new suture anchor of FIG. 13 loaded into a rotational driver and with a support sheath surrounding the proximal portion of the suture anchor;

FIGS. 30-34 are schematic views showing an alternative form of suture anchor, wherein the suture anchor comprises a wire trocar and an implant body;

FIGS. 35 and 36 are schematic views showing a tap loaded on the wire trocar of FIGS. 30 and 31;

FIGS. 37-43 are schematic views showing a “stab and drag” tendon repair using the suture anchor of FIGS. 30-34 and the tap of FIGS. 35 and 36; and

FIGS. 44-53 are schematic views showing a “partial tear” tendon repair using the suture anchor of FIGS. 30-34 and the tap of FIGS. 35 and 36.

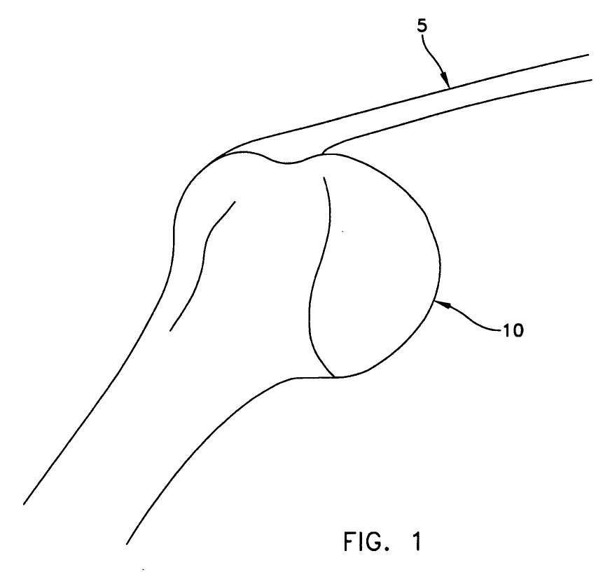

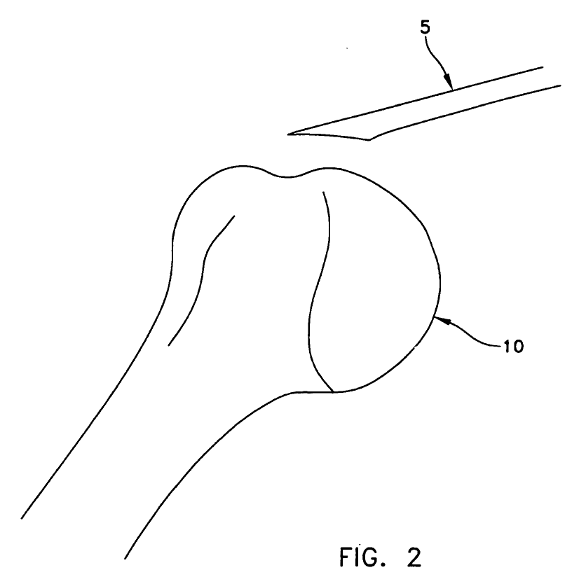

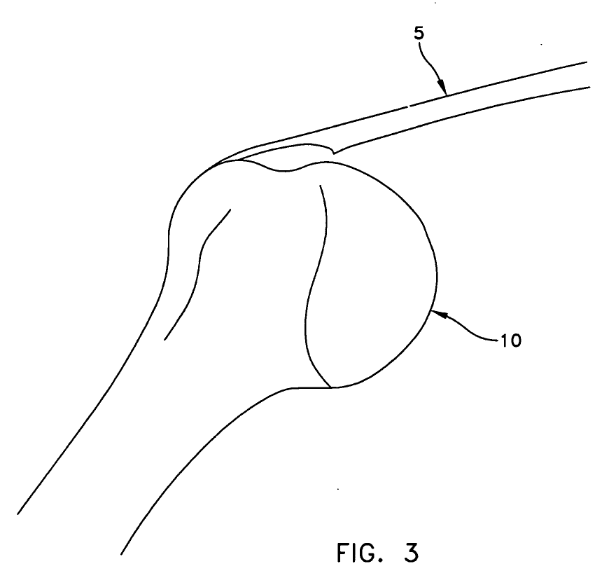

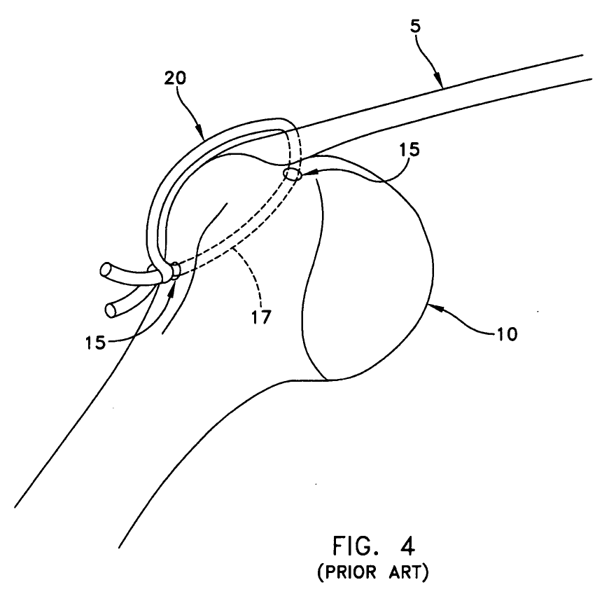

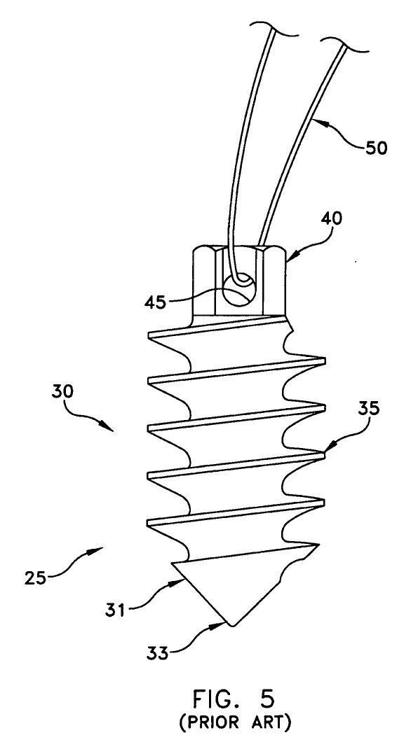

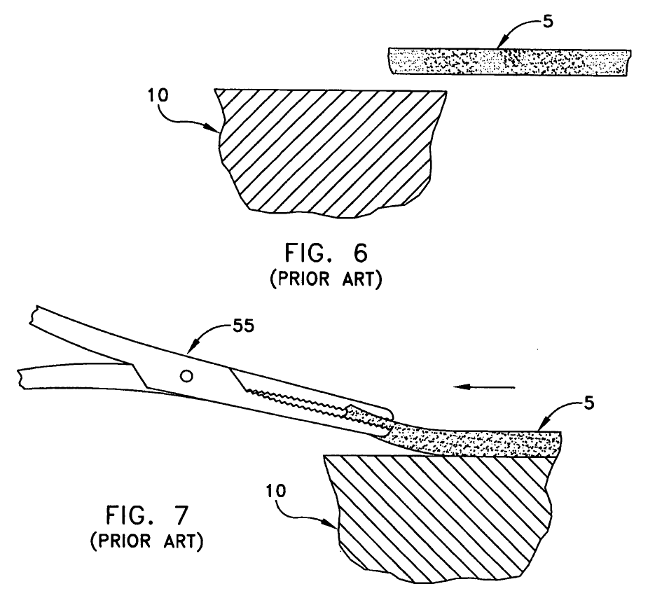

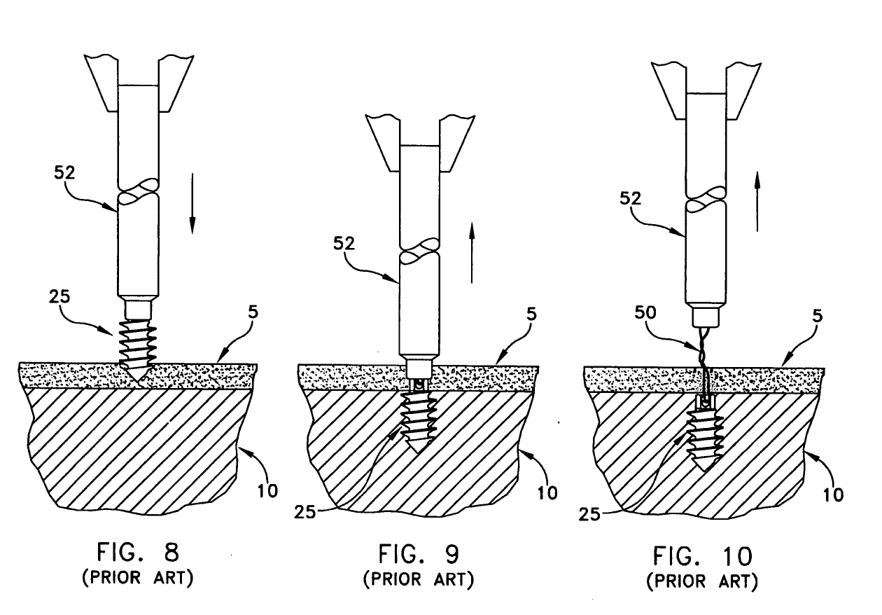

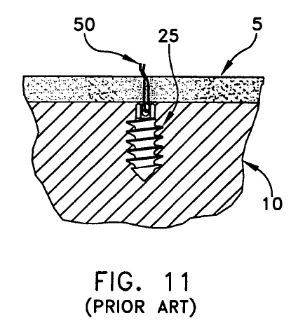

### DETAILED DESCRIPTION OF THE PREFERRED EMBODIMENTS

The present invention provides a new and improved method and apparatus for securing a tendon or ligament to a bone.

More particularly, the present invention provides a novel suture anchor and a novel method for re-attaching a tendon or ligament to bone using that novel suture anchor.

Even more particularly, the present invention comprises the provision and use of a novel suture anchor wherein the tip section of the suture anchor is significantly longer than normal, with a significantly increased distance between the distal point of the suture anchor and the start of the suture anchor's screw threads. In one preferred form of the invention, the tip section of the suture anchor is formed long enough such that the distal point of the suture anchor can be passed through a partially torn rotator cuff (RTC) tendon, or the full thickness of the rotator cuff (RTC) tendon, so that the distal point of the suture anchor can be seen protruding through the undersurface of the rotator cuff (RTC) tendon before the screw threads of the suture anchor have engaged the tendon or the bursal surface of the rotator cuff (RTC) tendon. For reference, it should be noted that the normal thickness of the terminal 2 cm of an intact rotator cuff (RTC) tendon generally ranges from between about 9 mm to about 12 mm in length. Thus, the new suture anchor of the present invention will preferably, but not necessarily, have a leading tip section (i.e., the distance between distal tip 33 and the start of screw threads 35) which is approximately 10 mm to 20 mm in length.

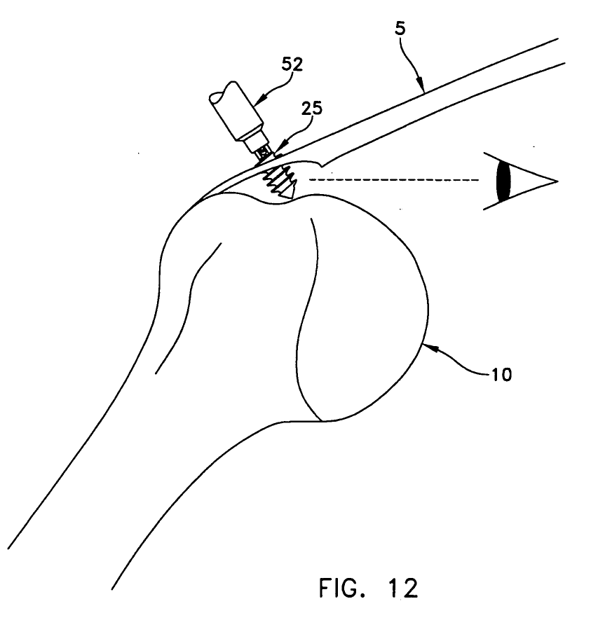

Looking now at FIG. 13, there is shown a new suture anchor 125 which comprises one preferred form of the invention, and which is particularly well suited for use in re-approximating a damaged rotator cuff (RTC) tendon to its insertion site on a humeral head. More particularly, the new suture anchor 125 comprises a body 130 having a tip section 131 terminating in a distal point 133 to facilitate passing the suture anchor through the tendon and introducing the suture anchor into a bone. Body 130 also has screw threads 135 for advancing the suture anchor into the interior of the bone, a hex-shaped (or other non-circular geometry, e.g., square, rectangular, Torx-type, etc.) rear end 140 for coupling the suture anchor to a rotary driver (not shown in FIG. 13), and an eyelet 145 for attaching a suture 50 to the suture anchor. Additional eyelets and/or sutures may also be provided.

In accordance with the present invention, tip section 131 is significantly longer than normal, with a significantly increased distance between distal point 133 and the start of the screw threads 135. In one preferred form of the invention, tip section 131 is formed long enough such that distal point 133 of suture anchor 125 can be passed through a partially torn rotator cuff (RTC) tendon, or the full thickness of the rotator cuff (RTC) tendon, so that distal point 133 can be viewed protruding through the undersurface of the rotator cuff (RTC) tendon before screw threads 135 have engaged the tendon or the bursal surface of the rotator cuff (RTC) tendon. Thus, in the case where the new suture anchor 125 is to be used to re-attach a damaged rotator cuff (RTC) tendon, suture anchor 125 will preferably have a leading tip section of approximately 10 to 20 mm in length, since the normal thickness of the terminal 2 cm of an intact rotator cuff (RTC) tendon generally ranges from between about 9 mm to about 12 mm in length.

The present invention also comprises a new method for re-attaching a tendon or ligament to bone using the new suture anchor of the present invention.

Thus, in one form of the invention, and looking now at FIGS. 14-19, there is provided a method of using the elongated tip section of the new suture anchor to spear the rotator cuff (RTC) tendon in such a way that the tendon can be dragged or repositioned or moved or otherwise re-approximated laterally back to the repair site (i.e., to the footprint on the humeral head) and then reseated to the bone without the need for a grasper instrument. In addition, the distal point of the suture anchor can be used as a “starting awl” or punch to aid in the placement of the suture anchor through the rotator cuff (RTC) tendon and into the bone.

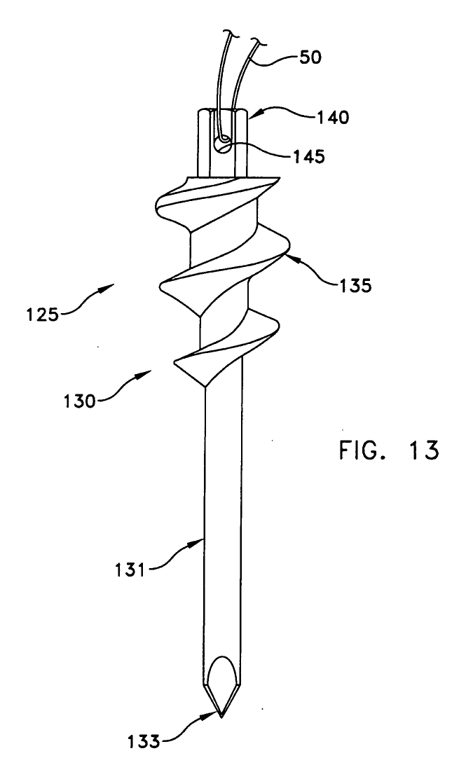

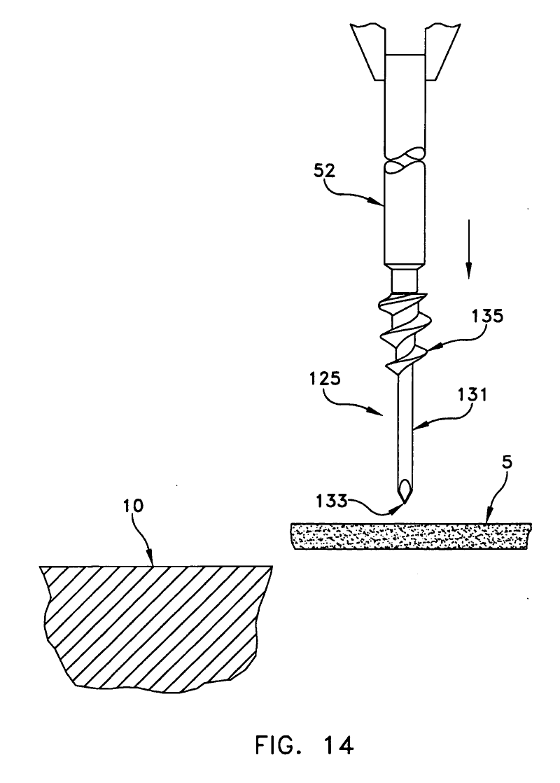

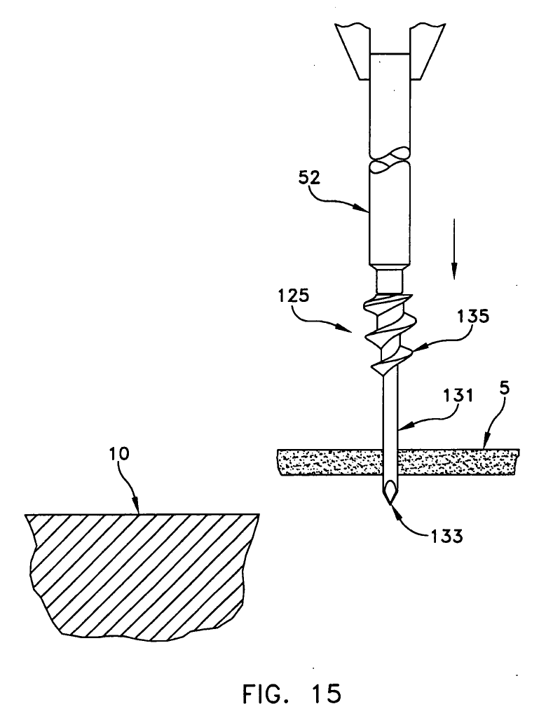

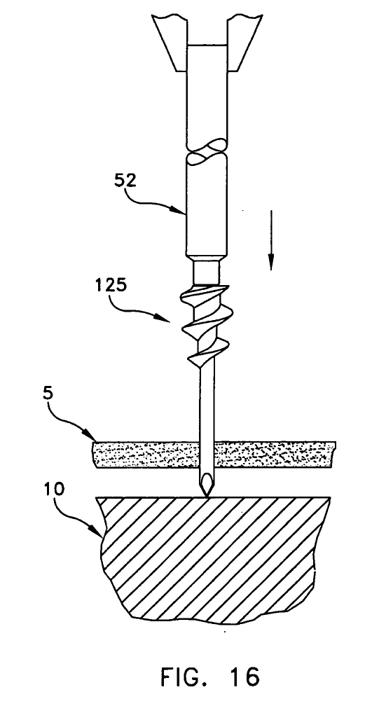

More particularly, in FIG. 14 there is shown a rotator cuff (RTC) tendon 5 which is completely torn away from humeral head 10, and the new suture anchor 125 mounted to a rotational driver 52. In accordance with one form of the present invention, the pointed distal tip 133 is “stabbed” through the rotator cuff (RTC) tendon 5 and the suture anchor is advanced distally so that the rotator cuff (RTC) tendon 5 is securely mounted on the suture anchor's elongated tip section 131 (FIG. 15). Then, using rotational driver 52 and suture anchor 125, the rotator cuff (RTC) tendon 5 is dragged laterally until the tendon is positioned over its footprint on humeral head 10, and then pointed distal tip 133 is positioned against the bone (FIG. 16). Then rotational driver 52 is used to advance suture anchor 125 into bone 10 (FIGS. 17 and 18), i.e., by rotating the suture anchor so that its screw threads 135 advance the suture anchor into the bone. Next, rotational driver 52 is disengaged from suture anchor 125 (FIG. 19) and then suture 50 is used to tie rotator cuff (RTC) tendon 5 down to humeral head 10.

In accordance with another form of the invention, the new suture anchor can be used to repair a rotator cuff (RTC) tendon which has been partially torn away from the humeral head. Significantly, when repairing partially torn rotator cuff (RTC) tendons with the new suture anchor, the surgeon can visualize the tear from the undersurface of the tendon (i.e., the articular side) while passing the new suture anchor from the superior surface of the tendon (i.e., the bursal side). The extended tip section of the new suture anchor can be used as a guide to decide where to place the suture anchor (e.g., in some ways analogous to the way one might use a spinal needle to identify a desired position for a bone anchor). This is because, due the novel construction of the suture anchor, the elongated tip section of the suture anchor is long enough that the distal point can be seen emerging from the undersurface of the tendon without having to advance the suture anchor so far that the suture anchor's threads engage the superior surface of the tendon.

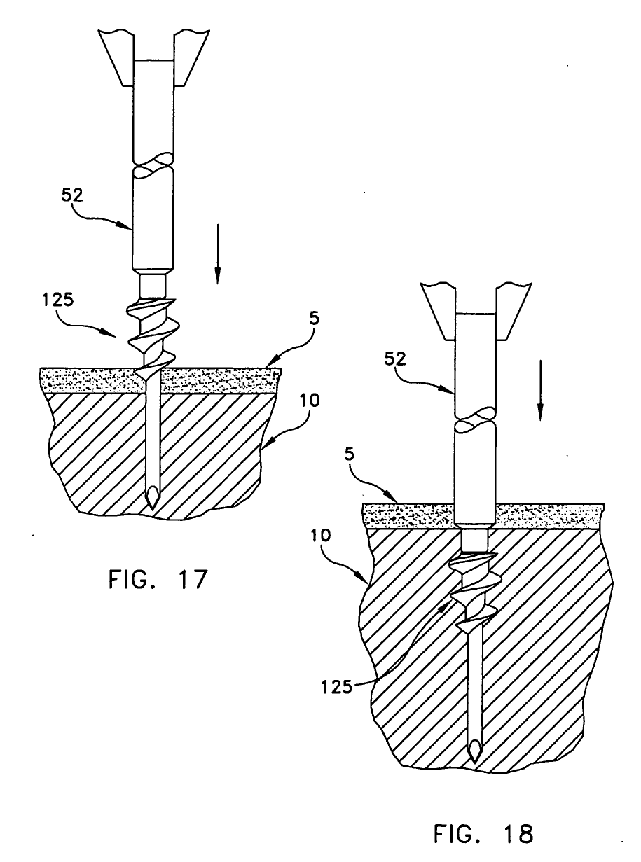

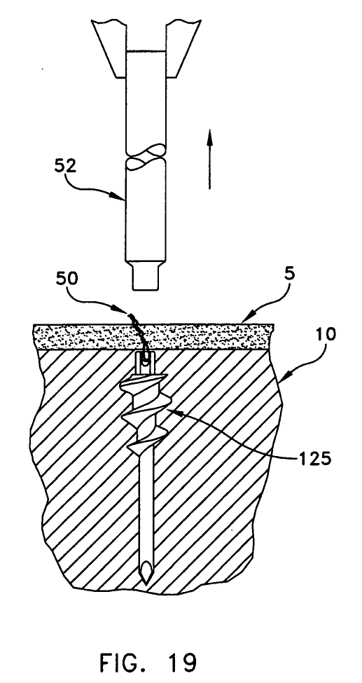

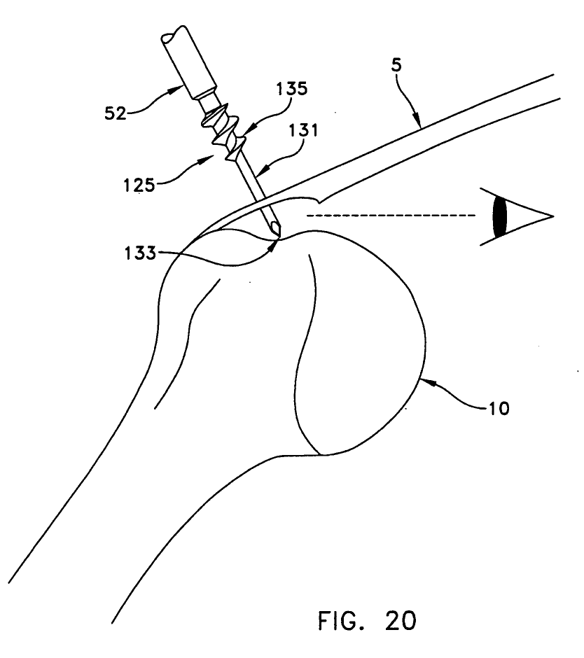

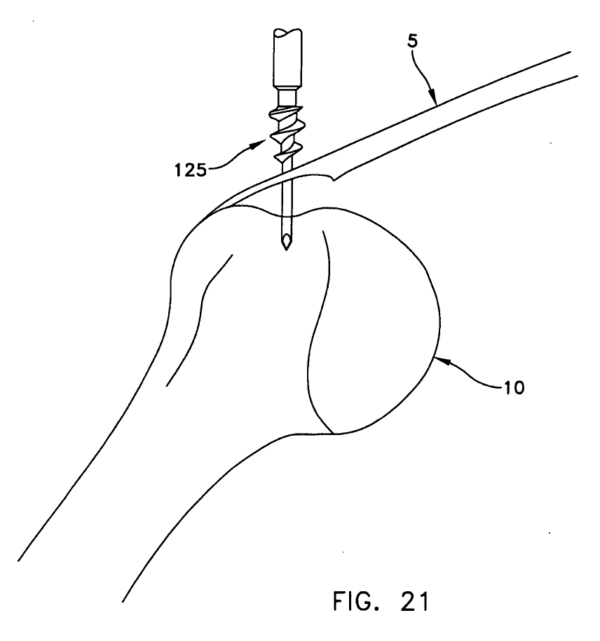

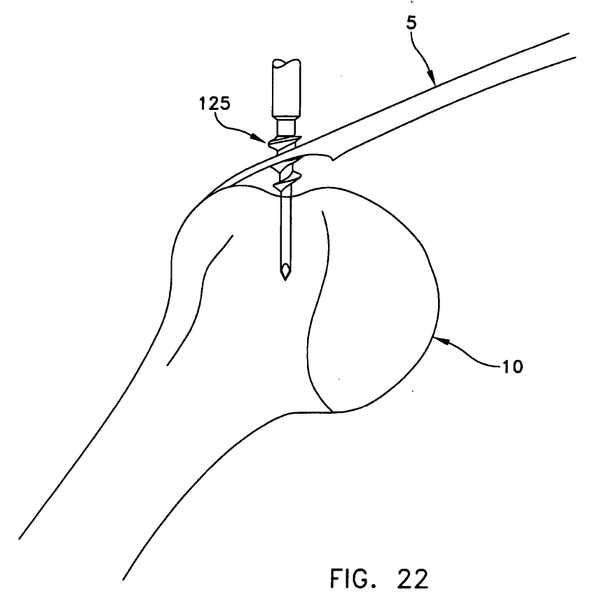

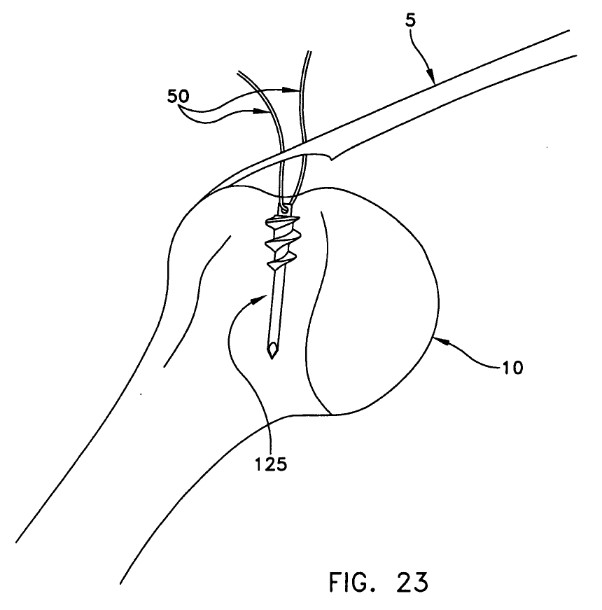

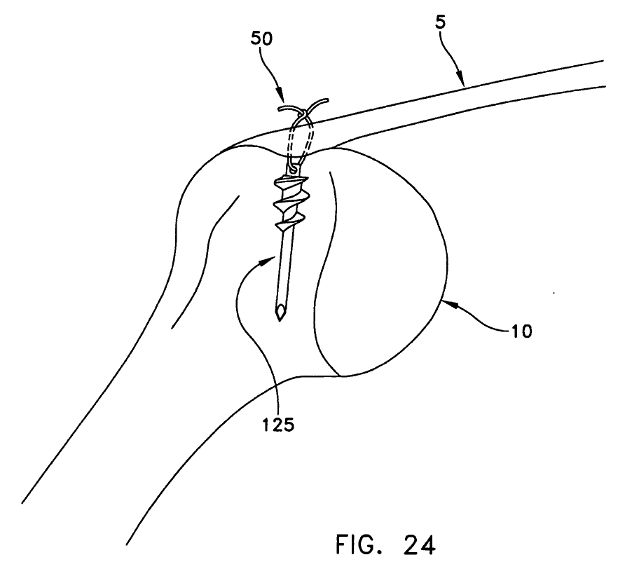

More particularly, and looking now at FIGS. 20-24), with this form of the invention, suture anchor 125 is advanced through rotator cuff (RTC) tendon 5 so that the distal point 133 is visible from the underside of the tendon but the screw threads 135 of the suture anchor have not yet engaged the top side of the tendon (FIG. 20). Then, while visualizing distal point 133, suture anchor 125 is properly positioned against humeral head 10 and then the suture anchor is advanced into the bone (FIGS. 21-23), i.e., by rotating the suture anchor so that its screw threads advance the suture anchor into the bone. Sutures strands 50 are then used to tie rotator cuff (RTC) tendon 5 into place against humeral head to (FIG. 24).

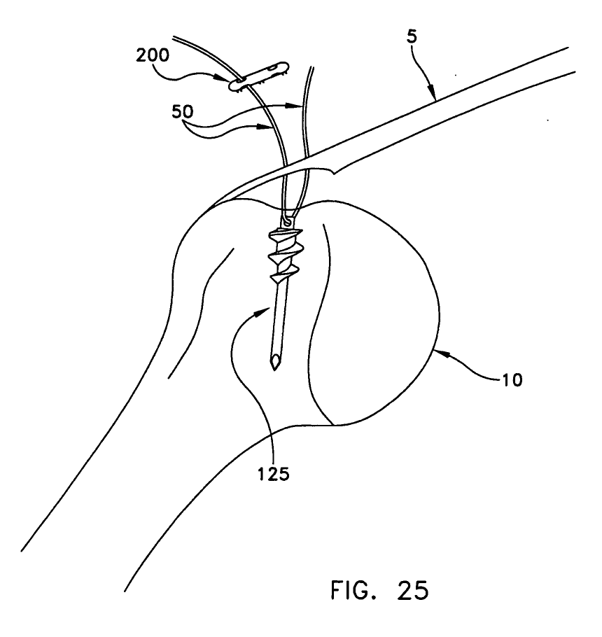

As noted above, once the suture anchor has been seated into bone, the suture 50 can be tied down to hold the rotator cuff (RTC) tendon against the humeral head. This can be done using various tie-down techniques well known in the art of suture anchors. Alternatively, and looking now at FIGS. 25 and 26, a device such as a button or sliding locking device 200 can mounted on the suture strands 50, slid down into place, thereby reducing the partially or fully torn tendon, and then locked or tied into position.

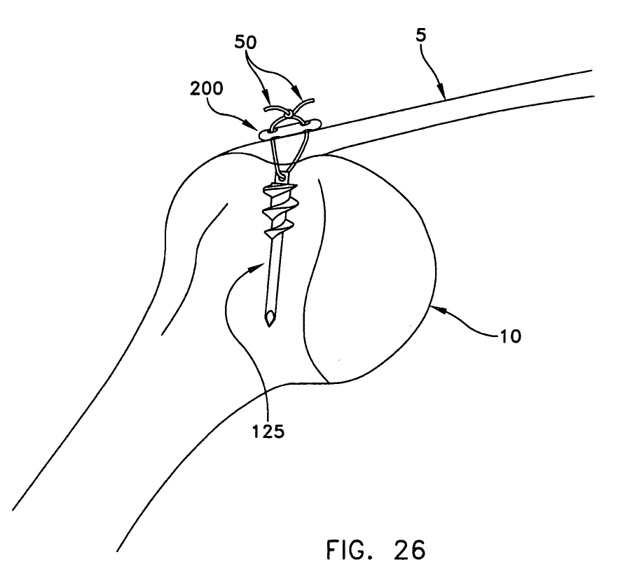

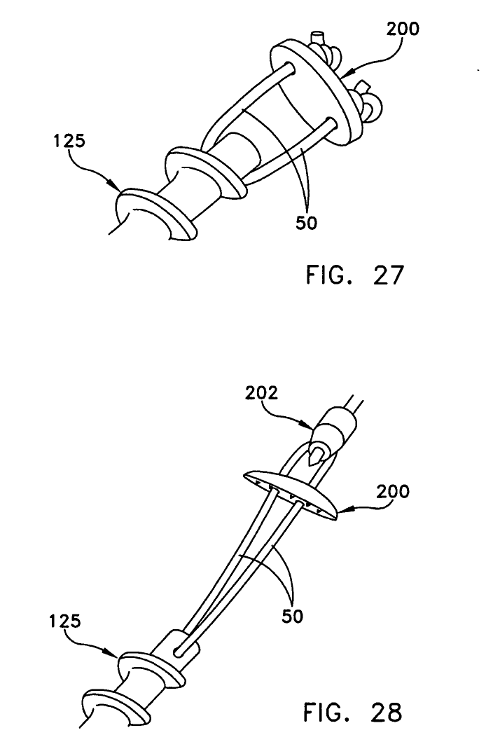

By way of further example, in FIG. 27, there is shown a button 200 connected to the suture anchor by suture 50, such that the button is spaced from a suture anchor by a substantially fixed length. With this construction, the tension holding the soft tissue to the bone is adjusted by varying the depth of insertion of the suture anchor into the bone.

By way of further example, in FIG. 28, there is shown a button 200 slidably mounted on suture 50, distal to a sliding knot 202. With this construction, the suture anchor is fully deployed into the bone, and then the sliding knot 202 is moved distally so as to force the button 200 into captivating engagement with the soft tissue.

As noted above, and as shown in FIGS. 14-19, the new suture anchor may be used to “stab and drag” a damaged rotator cuff (RTC) tendon so as to laterally move the displaced tendon back to its footprint. In such an application, it may be desirable to provide additional support to the proximal end of the suture anchor in order to accommodate the lateral loads imposed on the distal end of the suture anchor during such a “dragging” operation.

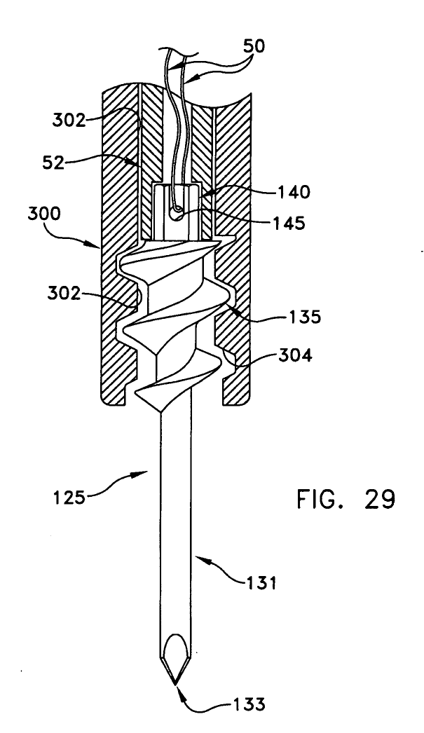

More particularly, as seen in FIGS. 14-18, the rotational driver 52 may have a distal end which engulfs the hex-shaped rear end 40 of suture anchor 125 and transmits rotational motion from driver 52 to suture anchor 125. However, as seen in these drawings, the rotational driver 52 does not engulf the screw threads 135. Therefore, and looking now at FIG. 29, there is shown a novel arrangement in which a support sheath 300 is disposed concentrically around rotational driver 52, with the distal end of the support sheath extending beyond the distal end of rotational driver 52. Support sheath 300 includes a smooth inner bore 302 for turnably receiving the exterior of rotational driver 52, and a helical recess 304 for turnably receiving the suture anchor's screw threads 135.

As a result of this construction, when the apparatus of FIG. 29 is used in a “stab and drag” procedure of the sort discussed above, the distal end of support sheath 300 provides lateral support to the proximal end of suture anchor 125 during the “drag” operation, with the support sheath's smooth inner bore 302 thereafter permitting the rotational driver 52 to turn, and therefore axially advance, suture anchor 125 out of the support sheath 300, through rotator cuff (RTC) tendon and into the bone.

As noted above, the extended tip section of the new suture anchor can be subjected to substantial lateral loads during the soft tissue repair, e.g., during the “stab and drag” operation. Where the new suture anchor is formed out of a metal material (e.g., stainless steel, titanium, etc.) or a strong non-metal material (e.g., a strong plastic, a strong absorbable material, etc.), the extended tip section may be strong enough to undergo such lateral loads without difficulty. However, in some circumstances (e.g., such as where the new suture anchor is to be formed out of certain absorbable materials), the extended tip section may not be strong enough or durable enough to safely withstand such lateral loads. In these circumstances, an alternative construction may be used.

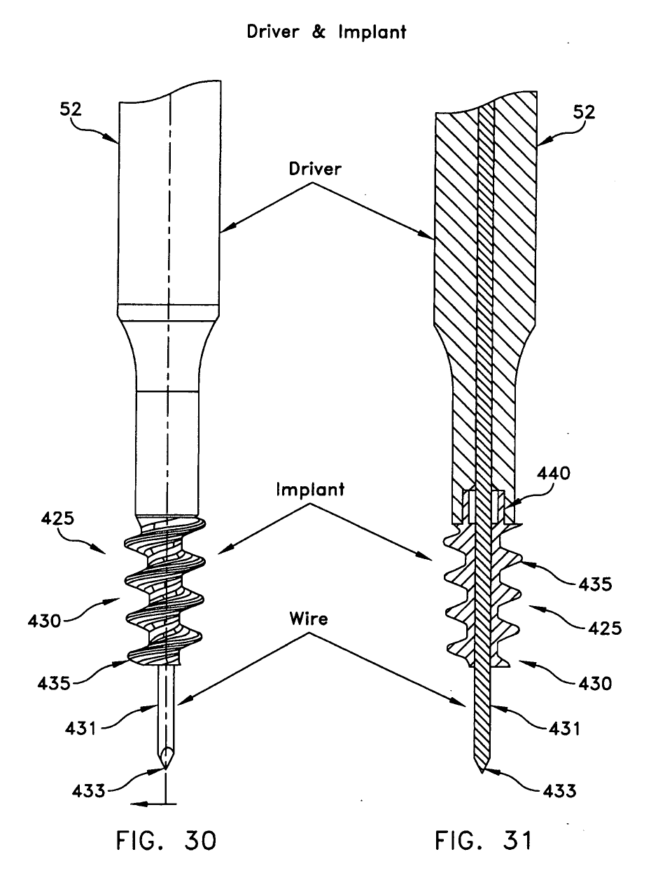

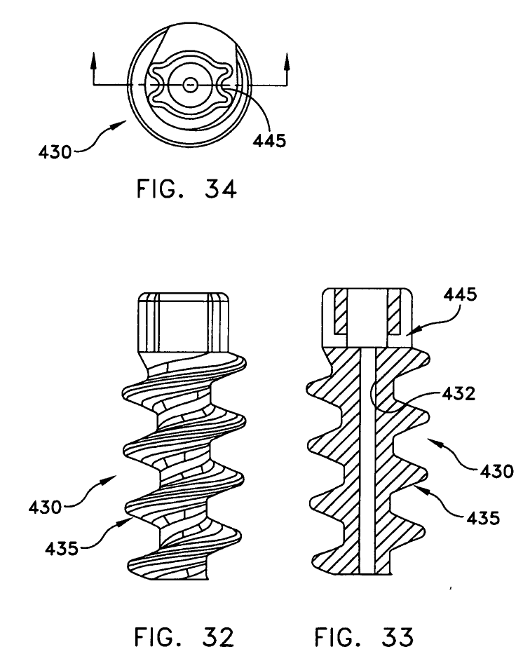

More particularly, and looking next at FIGS. 30-34, a new suture anchor 425 may be formed by a wire trocar 431 and an implant body 430. Wire trocar 431 terminates in a distal point 433. Implant body 430 is slidably disposed over wire trocar 431 and comprises a center bore 432 for receiving wire trocar 431 and screw threads 435 for advancing implant body 430 into bone. Implant body 430 terminates in a hex-shaped (or other non-circular geometry, e.g., square, rectangular, Torx-type, etc.) rear end 440 for connection to a rotational driver. Hex-shaped rear end 440 has an eyelet 445 for attaching a suture 50 thereto.

With this construction, wire trocar 431 and implant body 430 are unified during tendon penetration (and, in some cases, tendon “dragging”) and during the disposition of implant body 430 into the bone; wire trocar 431 is removed after implant body 430 is deployed in the bone, as will hereinafter be discussed. Furthermore, wire trocar 431 and implant body 430 are configured such that while they are so unified, the portion of wire trocar 431 extending beyond the distal end of implant body 430 is functionally equivalent to the elongated tip section 131 of suture anchor 125. Thus, the portion of wire trocar 431 extending beyond the distal end of implant body 430 will be longer than the thickness of the soft tissue which is to be re-attached to the bone, so that the sharp distal point 433 of wire trocar 431 will emerge from the bottom of the soft tissue before the screw threads 435 of implant body 430 engage the soft tissue.

In one form of the invention, the new suture anchor 425 may be used in substantially the same manner as the aforementioned suture anchor 125 (e.g., in the manner shown in FIGS. 14-19, or the manner shown in FIGS. 20-24, etc.), except that after deployment of implant body 430 into the bone, and before suture tie down, wire trocar 431 is removed.

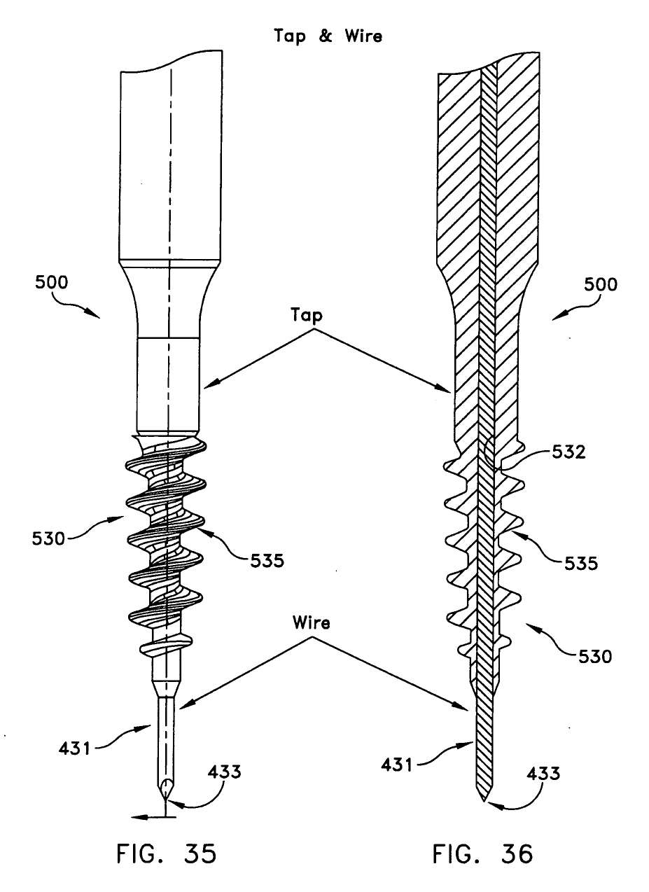

Alternatively, in view of the fact that, in this form of the invention, implant body 430 may be formed of a weaker or less durable material (e.g., a weaker absorbable material), it may be desirable to provide a tap 500 (FIGS. 35 and 36) for tapping the bone (i.e., so as to provide a screw thread seat) prior to disposition of implant body 430 within the bone. Tap 500 comprises a body 530 comprising a center bore 532 and screw threads 535. Center bore 532 of tap 500 slidably receives the wire trocar 431 of suture anchor 425, as will hereinafter be discussed.

Where a suture anchor 425 is to be used in conjunction with the tap 500, wire trocar 431 and tap 500 are unified during tendon penetration (and, in some cases, tendon “dragging”), and then wire trocar 431 and implant body 430 are unified during disposition of implant body 430 into the bone; and wire trocar 431 is removed after implant body 430 is deployed in the bone, as will hereinafter be discussed. Furthermore, wire trocar 431 and tap 500 are configured such that while they are so unified, the portion of wire trocar 431 extending beyond the distal end of tap 500 is functionally equivalent to the elongated tip section 131 of suture anchor 125. Thus, the portion of wire trocar 431 extending beyond the distal end of tap 500 will be longer than the thickness of the soft tissue to be re-attached to the bone, so that the sharp distal point 433 of wire trocar 431 will emerge from the bottom of the soft tissue before the screw threads 535 of tap 500 penetrate the soft tissue.

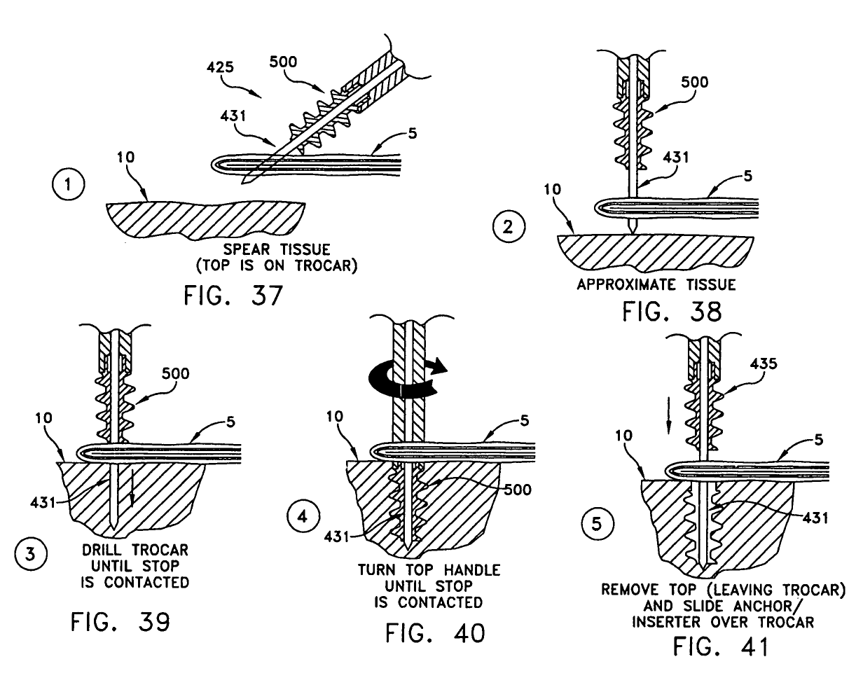

FIGS. 37-43 illustrate the new suture anchor 425 and tap 500 being used in a “stab and drag” tendon reconstruction procedure. More particularly, wire trocar 431 has tap 500 loaded thereon, with the distal tip section of the wire trocar extending well beyond the distal end of tap 500. More particularly, in accordance with the present invention, the distal tip section of the wire trocar extends sufficiently far beyond the distalmost portion of the tap so that the wire trocar can completely penetrate the tendon before the screw threads 535 of tap 500 penetrate the tendon. This assembly is used to “stab” rotator cuff (RTC) tendon 5 (FIG. 37), and then rotator cuff (RTC) tendon 5 is “dragged” into position over its footprint (FIG. 38). Next, wire trocar 431 is drilled into the humeral head 10 (FIG. 39), and then tap 500 is turned down into the bone so as to form a thread seat in the bone (FIG. 40). Then tap 500 is removed, leaving the wire trocar 431 in the anatomy.

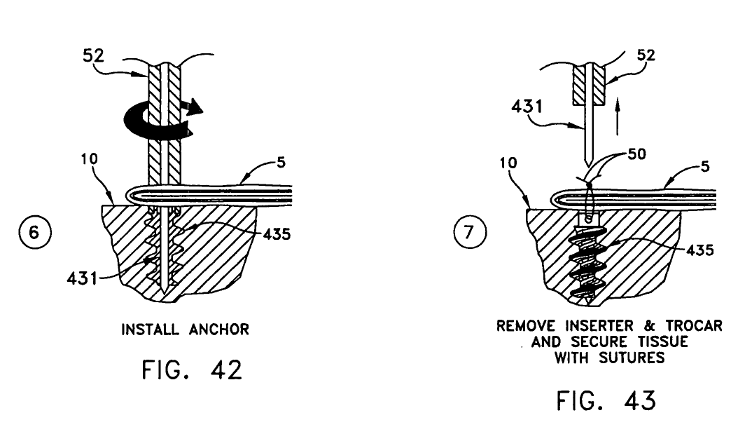

Next, implant body 435 is loaded onto the proximal end of wire trocar 431 and advanced down the wire trocar (FIG. 41). Then implant body 435 is turned into the bone using rotational driver 52 (FIG. 42), and then wire trocar 431 and rotational driver 52 are removed, leaving implant body 435 deployed in the bone (FIG. 43). Sutures 50 may then be used to tie down rotator cuff (RTC) tendon 5 into position at its footprint.

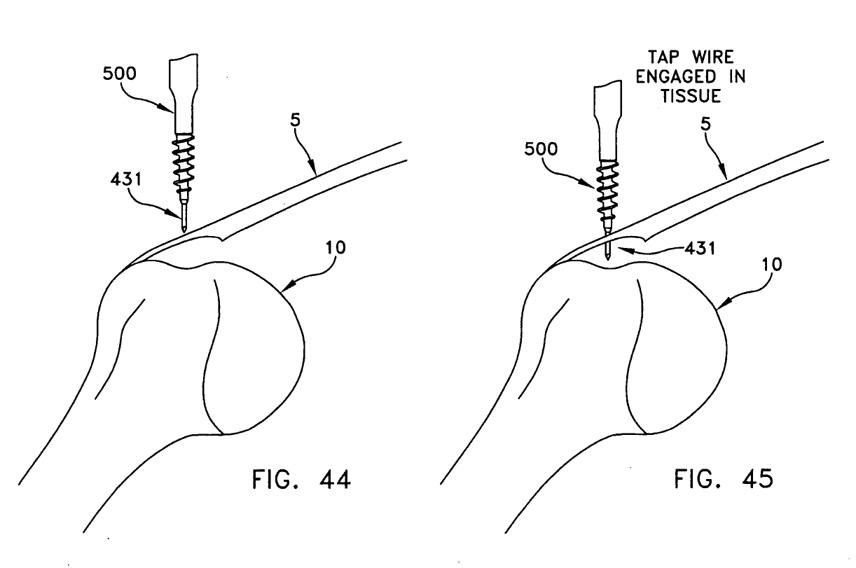

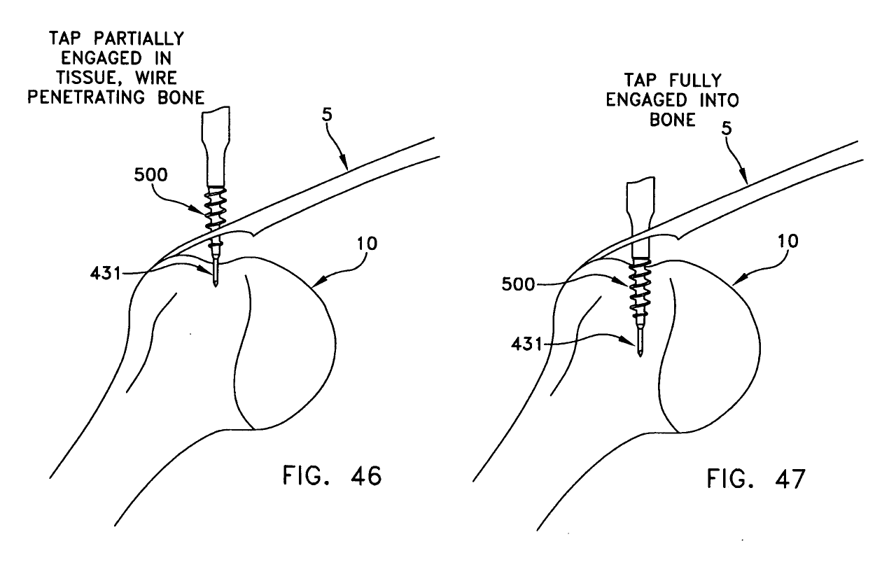

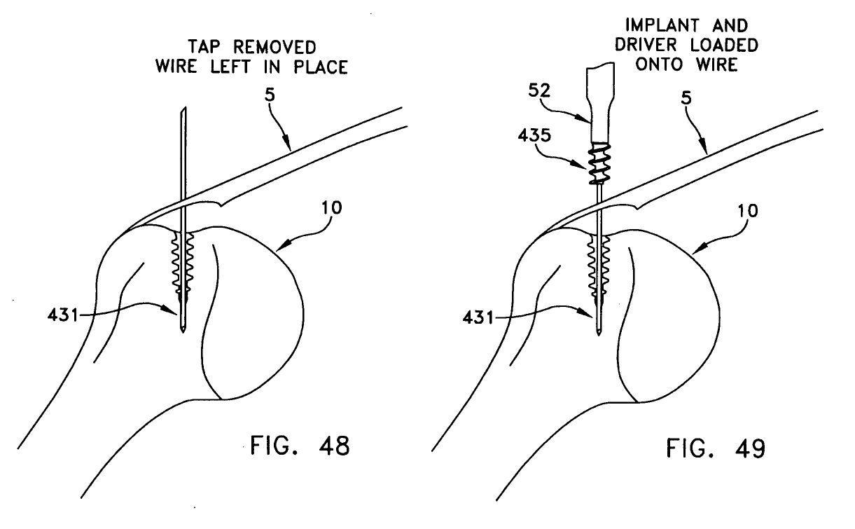

FIGS. 44-53 illustrate suture anchor 425 and tap 500 being used in a “partial tear” tendon repair. More particularly, wire trocar 431 has tap 500 loaded thereon, with the distal tip section of the wire trocar extending well beyond the distal end of tap 500, so that the wire trocar can completely penetrate the tendon before the screw threads 535 of tap 500 penetrate the tendon. This assembly is advanced through rotator cuff (RTC) tendon 5 and into the bone (FIGS. 44-47). In this respect it will be appreciated that inasmuch as the wire trocar 431 extends substantially beyond the distalmost portion of tap 500, the sharp point of the wire trocar 431 may be visualized as it emerges from the underside of the tendon and engages the top surface of the bone. Then tap 500 is removed from wire 431 (FIG. 48).

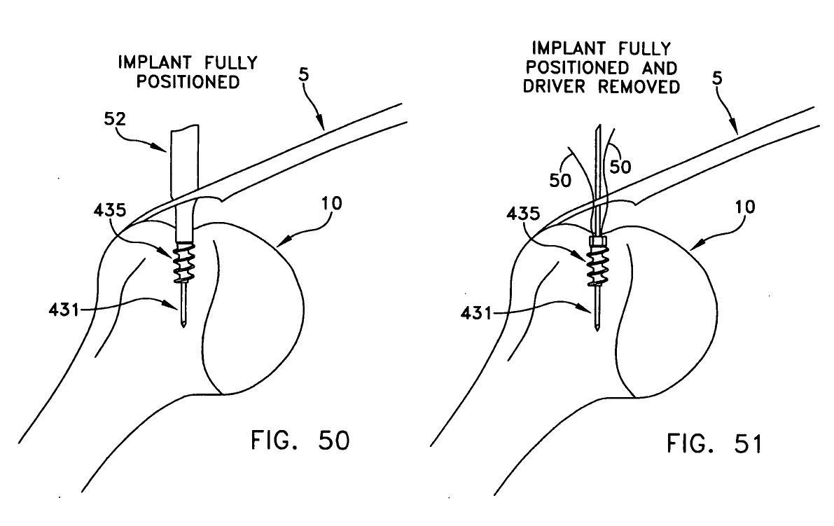

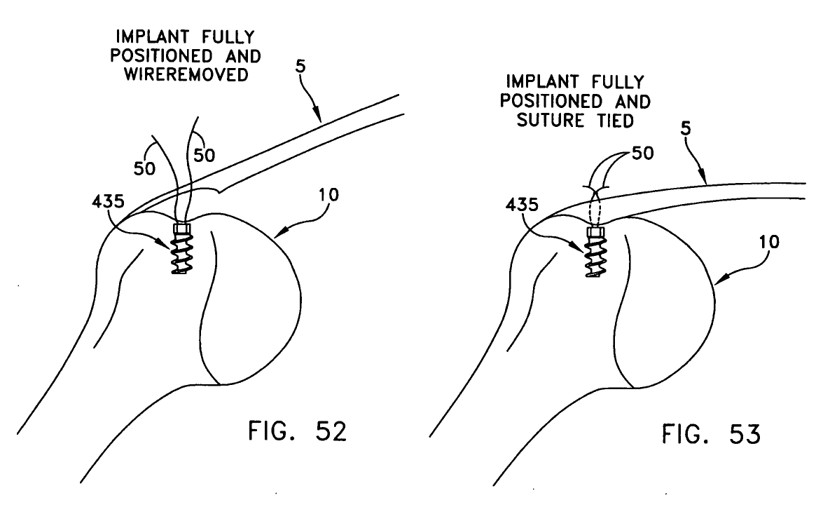

Next, implant body 435 is loaded onto the proximal end of wire trocar 431 and advanced down the wire (FIG. 49). Then implant body 435 is turned into the bone using rotational driver 52 (FIG. 50), and rotational driver 52 is removed, leaving wire trocar 431 and implant body 435 deployed in the bone (FIG. 51). Wire trocar 431 is then removed (FIG. 52). Sutures 50 may then be used to tie down tendon 5 into position (FIG. 53).

### MODIFICATIONS OF THE PREFERRED EMBODIMENTS

In addition to the foregoing, while in the foregoing description the present invention has been discussed in the context of a suture anchor employing screw threads, it is also possible to practice the invention with suture anchors not incorporating screw threads. Thus, the present invention can be practiced with suture anchors utilizing other bone-engaging geometries, e.g., the present invention can be practiced with rib-type suture anchors, barb-type suture anchors, etc.

Furthermore, while the constructions of FIGS. 35-53 are discussed in the context of a tap 500, the tap may be replaced by other bone-preparation devices consistent with the construction of the new suture anchor of the present invention, e.g., an awl, a dilator, a boring or acorn drill, etc.

It should also be appreciated that the present invention may be applied in the repair of anatomical structures other than the rotator cuff (RTC) tendon or ligament.

Furthermore, it should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for securing soft tissue to bone, comprising:
a suture anchor comprising:
a body;
an elongated tip section connected to the body and extending distally of the body, the elongated tip section terminating in a distal point;
a bone-engaging geometry formed on the body, the bone-engaging geometry being proximal to the elongated tip section;
a driver-engaging element formed on the proximal end of the body; and
a suture-connecting feature associated with the body for attaching a suture to the body;
wherein the elongated tip section has a length which exceeds the thickness of the soft tissue which is to be secured to the bone, such that when the suture anchor is passed through the soft tissue, the distal point emerges from the soft tissue before the bone-engaging geometry penetrates the soft tissue.

2. Apparatus according to claim 1 wherein the soft tissue comprises a tendon.

3. Apparatus according to claim 2 wherein the soft tissue comprises rotator cuff (RTC) tendon.

4. Apparatus according to claim 1 wherein the soft tissue comprises a ligament.

5. Apparatus according to claim 1 wherein the bone comprises a humerus.

6. Apparatus according to claim 1 wherein the suture anchor is formed out of metal.

7. Apparatus according to claim 6 wherein the suture anchor is formed out of stainless steel.

8. Apparatus according to claim 6 wherein the suture anchor is formed out of titanium.

9. Apparatus according to claim 1 wherein the suture anchor is formed out of plastic.

10. Apparatus according to claim 1 wherein the suture anchor is formed out of absorbable material.

11. Apparatus according to claim 1 wherein the suture anchor is formed out of a metal and a non-metal.

12. Apparatus according to claim 1 wherein the elongated tip section has a length which exceeds the full thickness of the rotator cuff (RTC) tendon.

13. Apparatus according to claim 1 wherein the elongated tip section has a length which exceeds the thickness of the detached portion of a partially torn rotator cuff (RTC) tendon.

14. Apparatus according to claim 1 wherein the elongated tip section has a length of approximately 10 to 20 mm in length.

15. Apparatus according to claim 1 wherein the bone-engaging geometry comprises screw threads.

16. Apparatus according to claim 1 wherein the bone-engaging geometry comprises ribs.

17. Apparatus according to claim 1 wherein the bone-engaging geometry comprises barbs.

18. Apparatus according to claim 1 wherein the driver-engaging element comprises a non-circular projection extending proximally.

19. Apparatus according to claim 18 wherein the non-circular projection has a hex-shaped cross-section.

20. Apparatus according to claim 18 wherein the non-circular projection has a square cross-section.

21. Apparatus according to claim 18 wherein the non-circular projection has a rectangular cross-section.

22. Apparatus according to claim 18 wherein the non-circular projection has a Torx-type geometry.

23. Apparatus according to claim 1 wherein the suture-connecting feature comprises an eyelet formed in the suture anchor.

24. Apparatus according to claim 1 wherein the suture anchor comprises a single integral structure.

25. Apparatus according to claim 1 wherein the suture anchor comprises an implant body and a wire trocar.

26. Apparatus according to claim 25 wherein the implant body comprises an axial bore and the wire trocar is selectively received within the implant body.

27. Apparatus according to claim 26 wherein the implant body comprises the body, the bone-engaging geometry, the driver-engaging element, and the suture-connecting feature, and wherein the wire trocar comprises the elongated tip section.

28. Apparatus according to claim 26 wherein, when the wire trocar is received within the implant body, the distance between the distal point and the distal end of the bone-engaging geometry exceeds the thickness of the soft tissue which is to be secured to the bone.

29. Apparatus according to claim 1 wherein the apparatus further comprises suture attached to the body.

30. Apparatus according to claim 1 wherein the apparatus further comprises a driver for inserting the suture anchor into bone.

31. Apparatus according to claim 30 wherein the driver comprises a support sheath for supporting the proximal portion of the suture anchor.

32. Apparatus according to claim 1 wherein the apparatus further comprises a tap.

33. Apparatus for securing soft tissue to bone, comprising:
a bone-preparation device having structure for forming a seat in a bone, the bone-preparation device having an axial bore; and
a wire trocar selectively received within the axial bore, the wire trocar comprising a distal point;
wherein, when the wire trocar is received within the bone-preparation device, the distance between the distal point and the distal end of the structure for forming a seat in a bone exceeds the thickness of the soft tissue which is to be secured to the bone.

34. Apparatus according to claim 33 wherein the bone-preparation device comprises a tap, and further wherein the structure for forming a seat in a bone comprises structure for forming a screw thread seat in the bone

35. Apparatus according to claim 33 wherein the apparatus further comprises:
an implant body comprising:
a body having an axial bore;
a bone-engaging geometry formed on the body;
a driver-engaging element formed on the proximal end of the body; and
a suture-connecting feature associated with the body for attaching a suture to the body.

36. A method for securing soft tissue to bone, comprising:
providing apparatus for securing soft tissue to bone, comprising:
a suture anchor comprising:
a body;
an elongated tip section connected to the body and extending distally of the body, the elongated tip section terminating in a distal point;
a bone-engaging geometry formed on the body, the bone-engaging geometry being proximal to the elongated tip section;
a driver-engaging element formed on the proximal end of the body; and
a suture-connecting feature associated with the body for attaching a suture to the body;
wherein the elongated tip section has a length which exceeds the thickness of the soft tissue which is to be secured to the bone, such that when the suture anchor is passed through the soft tissue, the distal point emerges from the soft tissue before the bone-engaging geometry penetrates the soft tissue;
advancing the suture anchor through the soft tissue so that the distal point emerges from the underside of the soft tissue before the bone-engaging geometry engages the soft tissue; and
advancing the suture anchor into the bone.

37. A method according to claim 36 wherein the suture anchor is used to move the soft tissue laterally before the suture anchor is advanced into the bone.

38. A method for securing soft tissue to bone, comprising:
providing:
apparatus for securing soft tissue to bone, comprising:
a bone-preparation device having structure for forming a seat in a bone, the bone-preparation device having an axial bore;
a wire trocar selectively received within the axial bore, the wire trocar comprising a distal point;
wherein, when the wire trocar is received within the bone-preparation device, the distance between the distal point and the distal end of the structure for forming a seat in a bone exceeds the thickness of the soft tissue which is to be secured to the bone; and
an implant body comprising:
a body having an axial bore;
a bone-engaging geometry formed on the body;
a driver-engaging element formed on the proximal end of the body; and
a suture-connecting feature associated with the body for attaching a suture to the body;
loading the bone-preparation device onto the wire trocar so that the distance between the distal point and the distal end of the structure for forming a seat in a bone exceeds the thickness of the soft tissue which is to be secured to the bone;
advancing the wire trocar through the soft tissue so that the distal point emerges from the underside of the soft tissue before the structure for forming a seat in the bone engages the soft tissue;
advancing the wire trocar into the bone, and advancing the bone-preparation device into the bone;
withdrawing the bone-preparation device from the bone;
loading the implant body onto the wire trocar and advancing it into the bone; and
withdrawing the wire trocar from the bone.
